(19) United States
(12) Patent Application Publication
(10) Pub. No.: US 2007/0009436 A1
(43) Pub. Date: Jan. 11, 2007

(54) Radionuclide nanoparticles encased by inorganic shell having vector biomolecules attached thereto

(76) Inventors: Adam J. Rondinone, Knoxville, TN (US); Sheng Dai, Knoxville, TN (US); Huimin Luo, Knoxville, TN (US); Suree S. Brown, Knoxville, TN (US)

Correspondence Address: AKERMAN SENTERFITT, P.O. BOX 3188, WEST PALM BEACH, FL, 33402-3188, US

(21) Appl. No.: 11/177,039

(22) Filed: Jul. 8, 2005

(51) Int. Cl. A61K 51/00; C07K 16/46

(52) U.S. Cl. 424/1.69; 977/906; 530/391.1

(57) ABSTRACT

A nanoparticle radionuclide delivery system includes a radionuclide core, a non-radioactive inorganic layer encasing the radionuclide core to form an encased radionuclide, at least one coupling moiety, and at least one vector biomolecule specific to a target tissue. The coupling moiety includes a first reactive group bound to the non-radioactive layer of the encased radionuclide and a second reactive group bound to the vector biomolecule. The vector biomolecule is a monoclonal antibody or fragment thereof, or a peptide having an affinity for the target tissue.

### STATEMENT REGARDING FEDERALLY SPONSORED RESEARCH OR DEVELOPMENT

The United States Government has rights in this invention pursuant to Contract No. DE-AC05-000R22725 between the United States Department of Energy and UT-Battelle, LLC.

### FIELD OF THE INVENTION

This invention relates generally to detection and treatment of cancer, and more particularly to radioimmunotherapy and imaging using radionuclide nanoparticles encased by an inorganic shell.

### BACKGROUND OF THE INVENTION

Radioimmunotherapy (RIT) is a therapeutic regimen used to treat diseases such as non-Hodgkins lymphoma (NHL). In RIT, a radioimmunoconjugate comprising a monoclonal antibody (mAb) that is specific to the target tissue of interest is bound to a radionuclide particle. The mAb attaches to a binding site on the target cell, and the radionuclide administers a lethal dose of radiation. RIT is used alone or can be combined with chemotherapy and other therapeutic strategies. The basic strategy of RIT is that coupling of a radionuclide to the mAb causes enhanced accumulation of the radionuclide at the targeted site. Accumulation of the radionuclide at the targeted site causes radiation therapy to be delivered near the targeted site with a radius approximating the mean path length of the emitted particle, about ˜5 mm for 2 MeV β particles from yttrium-90 (⁹⁰Y), and ˜100 μm for 8 MeV α particles from ²¹³Bi.

Effective treatment of cancer patients by using RIT and diagnostics of malignant tumors by radioimmunodetection (RAID) requires improvements of tumor-to-background (T/B) distribution of a radiolabel. The T/B ratio is the principal parameter that determines tumoricidal action, sensitivity of tumor detection and, most importantly, systemic toxicity of the compound. Radioactive isotopes of metals are used very often in various modalities of RIT and RAID in the form of conjugates with monoclonal antibodies.

Several different radionuclides have been considered for RIT therapies. The choice of radionuclide takes into account the physical and chemical characteristics of the radionuclide, including half-life, radiation emission properties, radiolabeling properties, availability, in vivo distribution and stability. Radionuclides considered suitable for RIT possess a half-life long enough for target localization, little or no gamma radiation, intermediate beta particle energy, stable daughter products, and stable fixation with an antibody system. Many β particle-emitting radionuclides are available for RIT. These include yttrium-90 (⁹⁰Y), iodine-131 (¹³¹I), copper-67 (⁶⁷Cu) and rhenium-186 (¹⁸⁶Re). Alpha (α) particle-emitting radionuclides include astatine-211 (²¹¹At), and bismuth-212 (²¹²Bi). Alpha and beta emitters are preferred because the mean path links are limited to dozens of mm, thereby limiting treatment to the immediate vicinity of the target. Beta particles may be more suitable for larger tumors due to the longer mean path length of the beta emission. Alpha particles generally have extremely high energies (greater than 5 MeV) and high linear energy transfer rates, which are useful for delivering high doses to a limited area.

The selection of the optimal target for RIT is one that expresses homogenously at high levels on the surface of the target cell, is absolutely or relatively specific to the cell in order to minimize toxicity to normal cells, and is not shed into the vascular compartment. Monoclonal antibodies have been developed against a number of B-cell antigens. CD20 is a particularly attractive target for RIT in the case of NHL. CD20 is expressed among 90% of B-cell NHL's, but is not expressed on stem cells, progenitor cells or fully differentiated plasma cells. This allows serum immunoglobulin levels to be maintained despite eradication of mature B cells, thus minimizing the potential for serious infection. CD20 is not shed from the cell surface, and is minimally internalized on antibody binding. Other RIT targets investigated for NHL includes CD19, CD22, and CD37, and HLA Class II variant antigens, which are selectively expressed on malignant B-cells.

Traditionally, the radionuclide is delivered as an atom bound directly to the mAb, such as ¹³¹I-mAb, or in a chelating complex bound to the antibody, such as ⁹⁰Y-DPTA-mAb. This approach, while effective, presents risk to the patient through unintentional release of the radionuclide as considerable leaching of the radionuclide metal ions is possible, which diminishes the practical applications of the RIT system.

Ideally, the mAb-radionuclide conjugate would be rapidly excreted via the urinary tract without intracellular retention, with intact conjugate accumulating and being retained selectively at the tumor site. This is however not always the case, and in several instances, persistent and unwanted retention of metallic nuclides has been reported.

The tumor uptake and overall biodistribution of the radiolabeled compound depends on the metabolism of the mAb and the strength of the radionuclide-antibody link. Although there is substantial room for improving targeting properties of mAbs, studies indicate that the thermodynamic and kinetic stability of the conjugate in vivo is of primary importance for the design of clinically successful radiochemicals.

Kotov, U.S. Pat. No. 6,689,338, discloses a bioconjugate including a radionuclide comprising nanoparticle covalently linked to a biological vector biomolecule. The particle core that contains the radionuclide is an inorganic crystal, such as a transition metal sulfide (e.g. ZnS) or oxide (e.g. TiO₂) that is doped throughout with the radionuclide. For example, the radionuclide ions can replace some of the parent metal (Zn) within the ZnS crystal structure. The radionuclide comprising core is then surrounded by a monolayer of an organic molecule which is covalently bound to the surface of the nanoparticle. The organic shell disclosed in Kotov tends to help passivate the surface of the nanoparticle and prevent reactivity and dissolution to some degree. However, the organic shell is not impermeable. Thus, although the organic shell disclosed by Kotov may reduce the unintentional release of the radionuclide due to leaching as compared to the conventional arrangements where no shell is used, the significant solubility of organic shell materials in biological fluids results in considerable leaching of the radionuclide metal ions, which diminishes the practical applications of the Kotov approach.

### SUMMARY OF THE INVENTION

A nanoparticle radionuclide delivery system based on a core-shell approach comprises a radionuclide core, a non-radioactive inorganic shell layer encasing the radionuclide core to form an encased radionuclide, at least one coupling moiety, and at least one vector biomolecule specific to a target tissue. The shell layer prevents radionuclide that may be at or near the surface of the radionuclide comprising core from dissolving away. The coupling moiety includes a first reactive group bound to the non-radioactive layer of the encased radionuclide and a second reactive group bound to the vector biomolecule. The vector biomolecule is generally a monoclonal antibody or fragment thereof or a peptide having an affinity for the target tissue.

The non-radioactive inorganic layer and the radionuclide core can share at least one chemical specie. In a preferred embodiment of the invention, the radionuclide core and the non-radioactive inorganic layer are part of a continuous crystal. For example, the non-radioactive inorganic layer can be Y₂O₃ and the radionuclide core can include ⁹⁰Y, such as in the form of ⁹⁰Y₂O₃. Oxide nanoparticles can be also synthesized with a percentage of ⁶²Zn—⁶²Cu (ZnO, CuO, or Cu₂O), ⁶⁸Ge—⁶⁸Ga (GeO₂ or Ga₂O₃), ⁶¹Cu/⁶⁴Cu, (CuO or Cu₂O), ¹⁷⁷Lu (Lu₂O₃), ¹⁵³Sm (Sm₂O₃), or ¹¹¹In (In₂O₃). The nanoparticle delivery systems can also be amorphous silica/alumina and TiO₂.

The coupling moiety can include at least one group bound to a protein provided by the vector biomolecule, such as 1-ethyl-3-[3-dimethylaminopropyl]carbodiimide hydrochloride. The nanoparticle radionuclide delivery system can further comprise an organic amine bound to the non-radioactive inorganic layer, wherein the 1-ethyl-3-[3-dimethylaminopropyl]carbodiimide hydrochloride is bound to the organic amine and the vector biomolecule. The organic amine can be 4-aminobenzoic acid or trimethoxysilylpropylamine. In this embodiment, the non-radioactive inorganic layer can comprise a metal oxide, such as yttrium oxide, or a metal phosphate, such as lanthanum phosphate (LaPO₄). The radionuclide core can comprise LaPO₄ and at least one radionuclide. The non-radioactive inorganic layer is preferably a continuous coating.

A method for treating or imaging tissue, comprises the steps of providing a plurality of encased radionuclide nanoparticles having vector biomolecules attached thereto comprising a radionuclide core, a non-radioactive inorganic layer encasing the radionuclide core to form an encased radionuclide, at least one coupling moiety, and at least one vector biomolecule specific to a target tissue, wherein the coupling moiety includes a first reactive group bound to the non-radioactive layer of the encased radionuclide and a second reactive group bound to the vector biomolecule. The plurality of encased radionuclide nanoparticles are infused into a patient in a dose selected to provide at least one of radioimmunotherapy and imaging of the tissue. The said vector biomolecule can be a monoclonal antibody or fragment thereof or a peptide having an affinity for the target tissue.

### BRIEF DESCRIPTION OF THE DRAWINGS

FIG. 1 is a schematic diagram of a nanoparticle-mAb compound according to the invention.

FIG. 2 is a schematic diagram illustrating the treatment of tumor cells with the nanoparticle radionuclide delivery system of the invention.

### DETAILED DESCRIPTION OF THE PREFERRED EMBODIMENTS

The invention provides a nanoparticle radionuclide delivery system including a radionuclide core, a non-radioactive inorganic layer encasing the radionuclide core to form an encased radionuclide, at least one coupling moiety, and at least one vector biomolecule specific to a target tissue. The coupling moiety includes a first reactive group bound to the non-radioactive layer of the encased radionuclide and a second reactive group bound to the vector biomolecule. The vector biomolecule is a monoclonal antibody or fragment thereof or a peptide having an affinity for the target tissue. As known in the art, monoclonal antibodies (mAb) are antibodies that are identical because they were produced by one type of immune cell, all being clones of a single parent cell. Given any substance, it is possible to create monoclonal antibodies that specifically bind to that substance.

The encasing layer is preferably a continuous coating. As used herein, the term “continuous coating” refers to a coating such that the radionuclide core is isolated from a physiological environment by the coating, such that the radionuclide core remains undissolved when the encased radionuclide is under physiological conditions for at least the length of time of a conventional radiotherapy session.

The radionuclide material is an atom or molecule that includes an atom with an unstable nucleus which undergoes radioactive decay by emitting a gamma ray(s) and/or subatomic particles. The radionuclide material can be any such material suitable for use in RIT or RAID. The radionuclide can be formed as a substantially homogeneous particle, or can be combined with one or more compounds. Such materials should emit radiation in sufficient amounts to be lethal to the target cell or provide a sufficient signal for imaging. It is preferable that the radiation from the radionuclide has a mean path length that is sufficiently limited (Betas are limited to ˜10-12 mm and Alphas are limited to ˜<1 mm) so as to minimize exposure of healthy cells to the emitted radiation. Suitable radionuclides can include a variety of alpha and beta emitters. Beta emitters may be better treatments for larger tumors due to the longer mean path link of the beta emitters. Alpha particles have extremely high energies and high linear transfer rates which may be useful for delivering high doses to a small area. Beta-emitting radionuclides include yttrium-90 (⁹⁰Y), iodine-131 (¹³¹I, indium-111, samarium-133, copper-67 (⁶⁷Cu) and rhenium-186 (¹⁸⁶Re). Alpha-emitting radionuclides include astatine-211 (²¹¹At), and bismuth-212 (²¹²Bi).

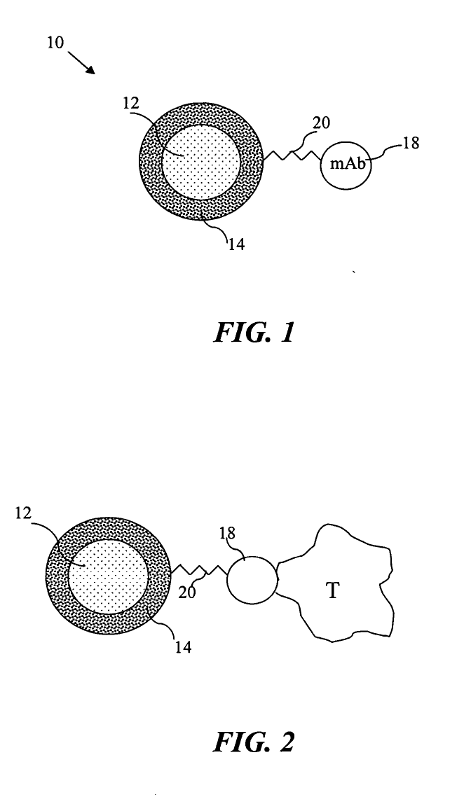

There is shown in FIG. 1 a radioimmunotherapy nanoparticle 10 comprising a core 12 which includes at least one radionuclide. The core is generally 50-100 Angstroms in size. The core 12 is encased by a continuous inorganic encasing layer 14. The encasing layer does not include radionuclide. The encasing layer is generally covalently bound to the core 12. The encasing layer should be at least one crystal unit cell in thickness, therefore at least about 7-8 Angstroms thick. In a preferred embodiment, the nanoparticles are on the order of <100 Angstroms, such as 40 to 60 Angstroms, which is approximately the same size as a typical mAb 18.

A biological vector biomolecule being a monoclonal antibody 18 is coupled to the encasing layer 14 by a coupling moiety 20. The coupling moiety 20 is generally covalent bound to the encasing layer 14. Although a single coupling moiety 20 is shown, the encased nanoparticle surface generally includes a plurality of covalently bound coupling moiety molecules 20. Similarly, although a single monoclonal antibody 18 is shown in FIG. 1, the encased nanoparticles generally include a plurality of vector biomolecules. In one embodiment of the invention, more than one different biological moieties, including other vector biomolecules, may be covalently linked to the encased nanoparticle in addition to the vector biomolecule to enhance its activity.

The material for inorganic encasing layer 14 is selected so as to be substantially impermeable to radionuclide particles, such that daughter particles will not escape the encasing layer. In a preferred embodiment, as noted above, the inorganic encasing layer is a continuous layer.

The encasing layer 14 is preferably a non-radioactive continuation of the core. For example, the core 12 may be Y₂O₃ isotopically enriched with Y-90. The encasing layer 14 would then be a layer of Y₂O₃ devoid of Y-90. Being a continuous crystal in this arrangement, the Y-90 would not be capable of migration. Yttrium oxide is easily doped with several useful radioactive elements other than Y-90. In another embodiment, a continuous crystal provides a lanthanum phosphate (LaPO₄) core 12 including the radionuclide, and an encasing layer 14 thereon of LaPO₄ that contains no radionuclide. LaPO₄ may be doped with any of the heavy lanthanide (III) ions, including actinium.

The encasing layer should be substantially stable in biological systems under human physiological conditions and when subjected to the radionuclide core material, should not be subject to leaching of the radionuclide, and should be capable of containing nuclear reaction products from the radionuclide. The encasing layer should be capable of facile attachment to various antibodies or selective ligands, without the need for expensive organic synthesis.

Oxide materials, such as metal oxides, are suitable for the inorganic encasing layer 14. Oxide materials are stable, especially under physiological conditions, and will not generally release free nuclides into the body. In addition, the protocol for the attachment of antibodies to oxide surfaces is well established. An atom encased in an oxide nanoparticle generally loses its atomic chemical nature. As noted above, in one embodiment, the non-radioactive inorganic encasing layer 14 and the core 12 share at least one chemical specie, such as Y₂O₃ and Y, respectively.

The non-radionuclide comprising nanoparticle encasement material can be of a variety of materials. Methods have been developed described in the Examples below for synthesizing oxide and phosphate encased nanoparticles with diameters of 50 nanometers or less. Yttrium, which in the ionic form (e.g. ZEVALIN® (ibritumomab tiuxetan) can accrete into bone, is instead utilized as yttrium oxide (Y₂O₃), which is a refractory and very insoluble ceramic material. Other oxide nanoparticles that can be employed as hosts for entrapment of targeted radionuclides include indium oxide, amorphous silica/alumina, zeolite, germanium oxide, gallium oxide, titanium oxide, iron oxide, rare-earth oxide, boron oxide, cupper oxide, cuprous oxide, zinc oxide, nickel oxide, and rhenium oxide. In another inventive aspect, the nanoparticles are rare-earth phosphate (e.g. lanthanum phosphate: LaPO₄) and aluminum phosphate (AlPO₄). Both yttrium oxide and lanthanum phosphate are highly insoluble under physiological conditions, and will contain both the radionuclide core 12 and any daughter products.

Yttrium oxide may be synthesized with a percentage or all of the yttrium as ⁹⁰Y. The above oxide nanoparticles can be also synthesized with a percentage of ⁶²Zn—⁶²Cu (ZnO, CuO, or Cu₂O), ⁶⁸Ge—⁶⁸Ga (GeO₂ or Ga₂O₃), ⁶¹Cu/⁶⁴Cu, (CuO or Cu₂O), ¹⁷⁷Lu (Lu₂O₃), ¹⁵³Sm (Sm₂O₃), or ¹¹¹In (In₂O₃). Lanthanum phosphate may be loaded with any rare earth nuclide and most trivalent transition metals. This includes alpha emitting radionuclides such as ²²⁵Ac that have not previously been considered because of the radiotoxicity of daughter products. For example, La₁₋ₓAcₓPO₄ will become La₁₋ₓAc_{x-y}Bi_{y}PO₄ as it decays, but the bismuth, the decay daughter of actinium, will remain in the nanoparticle and will not present a threat to other tissue.

The vector biomolecule 18 is preferably a monoclonal antibody (mAb). The monoclonal antibody can be any suitable monoclonal antibody which is capable of binding to the target. In one aspect, the monoclonal antibody is anti-CD20, such as mAb 2B8.

The nanoparticle may be bound to the vector biomolecule 18 using any suitable coupling moiety. In one example, the coupling moiety 20 is a protein coupler molecule such as EDC (1-ethyl-3-[3-dimethylaminopropyl]carbodiimide hydrochloride). EDC may couple an organic amine, such as 3-aminopropyltrimethoxysilane, that is bound to the surface of the oxide nanoparticle, to a carboxolate functionality within a protein provided by the vector biomolecule 18, such as glutamate or aspartate. The methoxysilane groups hydrolyze and react with hydroxyls on the surface of the yttria. The majority of the EDC molecule cleaves to leave an amide bridge between the amine and the protein. In this way, a nanoparticle mAb conjugate is formed.

It is also possible to add an imaging compound to the nanoparticles. Radioactive chemical tracers which emit radiation such as gamma rays can be included in nanoparticles according to the invention to provide diagnostic information about a person's anatomy and the functioning of specific organs. In the case of yttrium oxide encasing layers, a positron emitter such as ⁸⁷Y can be added to the particle to allow imaging. In the case of lanthanum phosphate, there are a variety of gamma emitters that may be used to add an imaging component to the treatment component.

A method for treating or imaging tissue according to the invention comprises the steps of providing a plurality of encased radionuclide nanoparticles having vector biomolecules attached thereto comprising a radionuclide core, a non-radioactive inorganic layer encasing the radionuclide core to form an encased radionuclide, at least one coupling moiety, and at least one vector biomolecule specific to a target tissue, wherein the coupling moiety includes a first reactive group bound to the non-radioactive layer of the encased radionuclide and a second reactive group bound to the vector biomolecule. A plurality of encased radionuclide nanoparticles are then infused into a patient in a dose selected to provide at least one of radioimmunotherapy and imaging. FIG. 2 shows a monoclonal antibody 18 bound to a surface receptor of the target cell 30. The radionuclide core 12 emits therapeutic radiation at a level that is lethal to the target cell 30 or radiation appropriate to provide diagnostic information about tissue surrounding cell 30, such as the functioning of a specific organ.

### EXAMPLES

It should be understood that the Examples described below are provided for illustrative purposes only and do not in any way define the scope of the invention.

Various methods may be used to form encased radionuclide nanoparticles having vector biomolecules attached thereto. Exemplary methods are described below for forming Y₂O₃ encased ⁹⁰Y and lanthanum phosphate encased trivalent lanthanide or actinide series species including ²²⁵Ac, a potent alpha emitting radionuclide.

In one method, yttrium oxide nanoparticles are synthesized using a polymer-complex method. Yttrium nitrate is mixed with malic acid in ethylene glycol to form a polymer complex. ⁹⁰Y may be introduced at this point as either the chloride or the nitrate salt. The ⁹⁰Y salt is incorporated into the polymer complex with the natural ⁸⁹Y. The polymer complex is thermally decomposed within a micelle to produce an amorphous Y₂O₃ nanoparticle, which is then heat-treated to produce the final particle. See Saengkerdsub, S; Im, H J; Willis, C; et al. Pechini-type in-situ polymerizable complex (IPC) method applied to the synthesis of Y₂O₃:Ln (Ln=Ce or Eu) nanocrystallites, Journal of Materials Chemistry, 14 (7): 1207-1211 2004) for exemplary process specifics.

The lanthanum phosphate nanoparticles can be synthesized through a direct reaction of lanthanum chloride within anhydrous phosphoric acid:
RCl₃+LaCl₃+H₃PO₄→3HCl+LaPO₄:R (R=²²⁵Ac or other radionuclide)

The reaction is performed at approximately 200° C. in order to promote crystallization and off gassing of the hydrogen chloride. The reactants are contained within a micelle in a manner identical to the yttrium oxide, in order to restrict the volume of the particles and prevent aggregation. Lanthanide or Actinide radioisotopes may be introduced into the micelles as chloride salts, and will be incorporated into the nanoparticles during the condensation with phosphoric acid. The nanoparticles may then be reacted with a coordinating organophosphate such as 2-aminoethylphosphonic acid. The organophosphate is reacted with ECD in the same manner as yttrium oxide to produce the LaPO₄ nanoparticle-mAb conjugate.

This invention can be embodied in other specific forms without departing from the spirit or essential attributes thereof. Accordingly, reference should be had to the following claims, rather than to the foregoing specification, as indicating the scope of the invention.

We claim:

1. A nanoparticle radionuclide delivery system, comprising:
a radionuclide core;
a non-radioactive inorganic layer encasing said radionuclide core to form an encased radionuclide;
at least one coupling moiety, and
at least one vector biomolecule specific to a target tissue, wherein said coupling moiety includes a first reactive group bound to said non-radioactive layer of said encased radionuclide and a second reactive group bound to said vector biomolecule.

2. The nanoparticle radionuclide delivery system of claim 1, wherein said vector biomolecule comprises a monoclonal antibody or fragment thereof or a peptide having an affinity for said target tissue.

3. The nanoparticle radionuclide delivery system of claim 1, wherein said radionuclide core and said non-radioactive inorganic layer are part of a continuous crystal.

4. The nanoparticle radionuclide delivery system of claim 1, wherein said non-radioactive inorganic layer and said radionuclide core share at least one chemical specie.

5. The nanoparticle radionuclide delivery system of claim 4, wherein said non-radioactive inorganic layer is Y₂O₃ and said radionuclide core includes ⁹⁰Y.

6. The nanoparticle radionuclide delivery system of claim 5, wherein said radionuclide core comprises ⁹⁰Y₂O₃.

7. The nanoparticle radionuclide delivery system of claim 1, wherein said coupling moiety includes at least one group bound to a protein provided by said vector biomolecule.

8. The nanoparticle radionuclide delivery system of claim 7, wherein said coupling moiety is 1-ethyl-3-[3-dimethylaminopropyl]carbodiimide hydrochloride.

9. The nanoparticle radionuclide delivery system of claim 8, further comprising an organic amine bound to said non-radioactive inorganic layer, wherein said 1-ethyl-3-[3-dimethylaminopropyl]carbodiimide hydrochloride is bound to said organic amine and said vector biomolecule.

10. The nanoparticle delivery system of claim 9, wherein said organic amine is 4-aminobenzoic acid or trimethoxysilylpropylamine.

11. The nanoparticle delivery system of claim 9, wherein said non-radioactive inorganic layer comprises a metal oxide.

12. The nanoparticle radionuclide delivery system of claim 11, wherein said metal oxide is yttrium oxide.

13. The nanoparticle radionuclide delivery system of claim 1, wherein said non-radioactive inorganic layer is lanthanum phosphate (LaPO₄).

14. The nanoparticle radionuclide delivery system of claim 13, wherein said radionuclide core comprises LaPO₄ and at least one radionuclide.

15. The nanoparticle delivery system of claim 1, wherein said non-radioactive inorganic layer is a continuous coating.

16. A method for treating or imaging tissue, comprising the steps of:
providing a plurality of encased radionuclide nanoparticles having vector biomolecules attached thereto comprising a radionuclide core, a non-radioactive inorganic layer encasing said radionuclide core to form an encased radionuclide, at least one coupling moiety, and at least one vector biomolecule specific to a target tissue, wherein said coupling moiety includes a first reactive group bound to said non-radioactive layer of said encased radionuclide and a second reactive group bound to said vector biomolecule, and
infusing said plurality of encased radionuclide nanoparticles into a patient in a dose selected to provide at least one of radioimmunotherapy and imaging of said tissue.

17. The method claim 16, wherein said vector biomolecule a monoclonal antibody or fragment thereof or a peptide having an affinity for said target tissue.

18. The method of claim 16, wherein said radionuclide core and non-radioactive inorganic layer are covalently bound.

19. The method of claim 16, wherein said non-radioactive inorganic layer and said radionuclide core share at least one chemical specie.

20. The method of claim 16, wherein said coupling moiety comprises a protein coupler.
